(19) United States
(12) Patent Application Publication
(10) Pub. No.: US 2024/0374338 A1
(43) Pub. Date: Nov. 14, 2024

(54) METHOD AND APPARATUS FOR MR-GUIDED FOCUSED UTLRASOUND

(71) Applicant: University of Maryland, Baltimore, Baltimore, MD (US)

(72) Inventors: Dheeraj Gandhi, Sparks, MD (US); Sijia Guo, Apex, NC (US); Rao Gullapalli, Woodbine, MD (US)

(21) Appl. No.: 18/658,177

(22) Filed: May 8, 2024

Related U.S. Application Data
(60) Provisional application No. 63/464,708, filed on May 8, 2023.

(51) Int. Cl. A61B 90/14

(52) U.S. Cl. CPC A61B 90/14

(57) ABSTRACT

An MR-compatible frame is configured for use in Magnetic Resonance (MR) guided focused ultrasound (MRgFUS) procedures. The frame is constructed from non-magnetic materials to ensure compatibility with MRI environments and to avoid interference with imaging quality and patient safety. The frame includes a hinge mechanism that allows for transitioning of the frame between open and closed states to enable quick and comfortable setup and removal around a patient's head. Adjustable components, including an adjustable back rest and adjustable, swivel-mounted front cushions enable customization to fit various head sizes and shapes to ensure a secure and stable positioning of the patient's head during procedures.

### CROSS-REFERENCE TO RELATED APPLICATIONS

This application claims the benefit of U.S. Provisional Patent Application No. 63/464,708 titled “ADJUSTABLE FRAME FOR MAGNETIC RESONANCE FOCUSED ULTRASOUND BACKGROUND,” filed by the inventors herein on May 8, 2023, the specification of which is incorporated herein by reference in its entirety.

### FIELD OF THE INVENTION

This invention relates generally to methods and apparatus for patient positioning and stabilization during medical imaging and therapeutic procedures, and more particularly to a pinless frame for positioning and stabilizing a patient's head during Magnetic Resonance Imaging (MRI) procedures and focused ultrasound therapies.

### BACKGROUND

Magnetic Resonance (MR) guided focused ultrasound (MRgFUS) is a rapidly advancing technology for treating a variety of central nervous system pathologies, including neurodegenerative, functional, and malignant disorders. It is an attractive alternative to brain penetrating procedures because this technique can be used to create precise, incisionless, transcranial lesions. Reliable, transient blood brain barrier (BBB) opening and drug delivery can also be achieved with the combination of MRgFUS and IV injection of ultrasound contrast agents to deliver a range of therapeutic agents. Moreover, low intensity FUS is being investigated as a brain mapping and neuromodulation tool for numerous neurologic diseases, such as by way of non-limiting example chronic pain, Alzheimer's disease, dementia, and Parkinson's disease.

At the present time, FUS procedures require firm stabilization of the patient's head to achieve the required high degree of precision and accuracy, using affixation of a stereotactic head frame with pins that penetrate the patient's scalp and fix to the outer table of the patient's skull. An alternative frame that uses dental mold assembly (DMA) is available, but cannot be used universally for long procedures due to the need for active patient involvement and for patients that are unable to co-operate (e.g., pediatric and unconscious patients or those with dental conditions that may make this difficult).

Stereotactic frame placement is typically uncomfortable and even at times painful, and carries a risk of scalp hematoma, black eye, and infection. The process of placing a stereotactic frame often evokes anxiety and discomfort amongst most patients. Although it is uncommon, a small fraction of patients are even unable to tolerate frame placement, the necessary first step for MRgFUS procedures. Frame placement is especially challenging in patients with chronic pain syndromes that suffer from sensory hypersensitivity.

When a patient is to undergo MRgFUS, the patient's head is shaved and affixed to the stereotactic head frame. This involves anesthetizing the skin, soft tissues and periosteum followed by affixation of the frame to the head with pins. Fixation to a stereotactic head frame and subsequently on to the focused ultrasound transducer is necessary in order to ensure lack of motion of the head during the ablative therapy with FUS. During the procedure, chilled, degassed water is circulated around the patient's head. The ultrasound beam alignment is performed using MR thermometry at low energy and temperature levels (45 degrees or so). As the acoustic energy is increased, the clinical effects start becoming evident when the target temperature approaches 50 degrees. However, higher temperatures (55-60 degrees) are required for ablative therapies that result in irreversible coagulative necrosis and permanent clinical effect.

Any subject motion during a sonication or between sonications can increase the risk of a significant adverse event. Subject motion can result in movement of the tissue relative to the planned treatment target. In extreme cases, this could result in the treatment of a volume outside the planned treatment target. While the use of a stereotactic frame is an inherent and essential component of the ablative therapy with MRgFUS, it also presents a major hindrance in adapting this technology for non-ablative applications such as neuromodulation and blood brain barrier disruption. Moreover, stereotactic frames, once positioned, offer limited flexibility for adjustment, which can be problematic in long or complex treatments.

Non-ablative applications require a tiny fraction of the acoustic power that is necessary in ablative procedures and generate negligible temperature rise. For example, neuro-stimulatory studies use relatively low pressures (<0.6 MPa at focus) and result in hardly discernable temperature increases (≤0.01° C.) (7, 8). Another important aspect of neuromodulation is that the focal spot need not be as precise as in ablative treatments and hence the requirement for immobility, while still important, is not as rigid in the ablative procedure scenario. Given the safety of these FUS parameters, a stereotactic frame is not necessary and therefore, there is a need in the art for a device/head restraint system that can curtail head motion without the need for scalp and periosteal penetration.

Additionally, innovative trial protocols and treatments must be constrained, limited, and in some cases abandoned due to the inherent impracticality of placing a head frame or lack of universal applicability of a DMA frame. For example, repeated sonication sessions (including daily or weekly treatments) may be necessary for some indications, such as neuromodulation for pain, depression and drug addiction. Frequent treatment sessions are also necessary for blood brain barrier opening in the treatment of disorders such as infiltrating gliomas and Alzheimer's disease. Moreover, broad acceptance and application of MRgFUS are unlikely, especially for uses related to brain mapping in normal volunteers, preoperative testing for prediction of procedural success or neuromodulation, unless an alternative fixation method is made available.

### SUMMARY OF THE INVENTION

Provided herein according to several exemplary configurations is an MR-compatible, adjustable frame that avoids one or more of the foregoing disadvantages of traditional stereotactic frames. A frame according to certain aspects of the invention provides a noninvasive alternative to traditional stereotactic frames, incorporating a pinless design configured to securely encircle the patient's head without the need for penetrating the scalp or periosteum, thereby eliminating the associated discomfort and risks. A frame configured in accordance with aspects of the invention also includes adjustable components, including back rest support that are adjustable side-to-side and include a cushion for added patient comfort, with adjustment being facilitated by a clamp mechanism, allowing for easy and precise positioning. Likewise, front pads are attached to adjustable front posts, which can be modified in height and along a width of the patient's head to fit different head sizes and shapes. The pads may include a swivel mechanism to adjust the angle of the front pads relative to the front posts, ensuring a secure and comfortable fit against the patient's head. The frame body comprises a hinged assembly that allows it to transition between an open state and a closed state, which facilitates easy placement and removal of the frame, which is particularly beneficial for patients who are claustrophobic or unable to fully cooperate during the procedure. A locking insert at the front of the frame body is configured to secure the hinged body portions in the closed state without the use of traditional screws and pins, further simplifying the setup and removal process. The frame is formed from non-magnetic materials, such as aluminum, Nylon, and other suitable composites to ensure MR compatibility. These materials are chosen for their non-magnetic properties, durability, and lightweight nature, which is critical for maintaining image quality and patient safety in an MRI environment.

An MR-compatible frame for focused ultrasound procedures configured in accordance with aspects of the invention may provide advancements over prior head fixation systems, and more particularly traditional stereotactic frames. Particularly, such a frame may eliminate the need for invasive techniques for attachment to the patient by using a pinless design that enhances patient comfort and safety by reducing the risk of complications associated with scalp penetration. Further, such a frame offers adjustable components, such as a back rest and front cushions, which can be easily modified to fit various head sizes and shapes. This adjustability ensures a more comfortable and secure fit, which is crucial for both imaging and therapeutic procedures. Still further, such a frame may be constructed from non-magnetic materials, such as aluminum and Nylon, ensuring that it does not interfere with the MRI's magnetic field or imaging quality, which is critical for obtaining clear images and effective treatment delivery during MR-guided procedures. Still further, such a frame provides a locking mechanism enabling transitioning of the frame from an open state to a closed state, easing the process of securing the frame on a patient and allowing for quick disengagement when necessary.

In accordance with certain aspects of an embodiment of the invention, an MR-compatible frame is provided for use in MR-guided focused ultrasound procedures, comprising: an ovoid body having a rear arm, a front left arm, and a front right arm; a first hinge affixing a first end of the front left arm to a first end of the rear arm; a second hinge affixing a first end of the front right arm to a second end of the rear arm; and a locking insert configured to simultaneously engage a second end of the front left arm and a second end of the front right arm. The first hinge and the second hinge may be configured to transition the frame between an open state and a closed state, and the frame may be formed from non-magnetic materials. Opposite ends of the locking insert may each define an interlocking member shaped to engage with a mating interlocking receiver on each of the second end of the front left arm and the second end of the front right arm.

The MR-compatible frame may further comprise: one or more adjustable hind posts; and a back rest mounted to the one or more adjustable hind posts; wherein the back rest is adjustable on the hind post from side to side. The one or more adjustable hind posts may likewise be adjustable in height from the ovoid body. Further, the back rest may have a window extending through the back rest, wherein a back rest clamp moveably engages the window to enable releasable and selective positioning of the back rest with respect to the one or more adjustable hind posts.

The MR-compatible frame may further comprise: a first front post adjustably mounted to the front hinged right arm, and a first front cushion attached to the first front post; and a second front post adjustably mounted to the front hinged left arm and a second front cushion attached to the second front post; wherein each of the first front post and the second front post is adjustable in height from the ovoid body. Each of the first front cushion and the second front cushion may be attached to the first front post and the second front post, respectively, via a swivel attachment.

The MR-compatible frame may further comprise a mounting bracket attached to an outer edge of the ovoid body, wherein the mounting bracket is configured for locking engagement with a patient support table.

In accordance with further aspects of an embodiment of the invention, a method for immobilizing a patient's head during an MR-guided focused ultrasound procedure is provided, comprising the steps of: positioning a patient's head in an MR-compatible frame, wherein the MR-compatible frame comprises: an ovoid body having a rear arm, a front left arm, and a front right arm; a first hinge affixing a first end of the front left arm to a first end of the rear arm; a second hinge affixing a first end of the front right arm to a second end of the rear arm; and a locking insert configured to simultaneously engage a second end of the front left arm and a second end of the front right arm; and after positioning the patient's head in the MR-compatible frame, adjusting the frame from an open state to a closed state by closing the front left arm and the front right arm around a front of the patient's head. The frame may be formed from non-magnetic materials.

The method may further comprise the step of: after adjusting the frame from an open state to a closed state, engaging the locking insert with the second end of the front left arm and the second end of the front right arm to lock the frame in the closed state about the patient′ head.

The frame may further comprise a back rest movably mounted to the frame, and the method may further comprise the step of adjusting a height of the back rest with respect to the ovoid body. The method may also further comprise the step of adjusting a position of the back rest from side to side.

The frame may further comprises a plurality of front cushions movably mounted to the frame, and the method may further comprise the step of adjusting an angle of a radially interior face of each front cushion with respect to the ovoid body. The method may further comprise the step of adjusting a height of each front cushion with respect to the ovoid body.

The ovoid body may further comprise a mounting bracket attached to an outer edge of the ovoid body, and the method may further comprise the step of temporarily affixing the frame to a patient support table.

Still other aspects, features and advantages of the invention are readily apparent from the following detailed description, simply by illustrating a number of particular embodiments and implementations, including the best mode contemplated for carrying out the invention. The invention is also capable of other and different embodiments, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized. The present invention is illustrated by way of example, and not by way of limitation, in the figures of the accompanying drawings, in which like reference numerals refer to similar elements, and in which:

FIG. 1 is a front perspective view of an MR-compatible, adjustable frame according to certain aspects of an embodiment of the invention.

FIG. 2 is a front view of the MR-compatible, adjustable frame of FIG. 1.

FIG. 3 is a side view of the MR-compatible, adjustable frame of FIG. 1.

FIG. 4 is a top view of front cushions for use on the MR-compatible, adjustable frame of FIG. 1.

FIG. 5 is a rear perspective view of the MR-compatible, adjustable frame of FIG. 1.

FIG. 6 is a rear perspective view of the MR-compatible, adjustable frame of FIG. 1 positioned about a patient's head.

FIG. 7 is a side view of the MR-compatible, adjustable frame of FIG. 1 attached to a neck brace according to further aspects of an embodiment of the invention.

FIG. 8 is a diagrammatic overview of an experimental design used for testing a prototype MR-compatible, adjustable frame configured in accordance with aspects of the invention.

FIG. 9 provides results from the testing of a prototype MR-compatible, adjustable frame configured in accordance with aspects of the invention.

### DETAILED DESCRIPTION OF THE PREFERRED EMBODIMENTS

The following detailed description is provided to gain a comprehensive understanding of the methods, apparatuses and/or systems described herein. Various changes, modifications, and equivalents of the systems, apparatuses and/or methods described herein will suggest themselves to those of ordinary skill in the art.

Descriptions of well-known functions and structures are omitted to enhance clarity and conciseness. The terminology used herein is for the purpose of describing particular embodiments only and is not intended to be limiting of the present disclosure. As used herein, the singular forms “a”, “an” and “the” are intended to include the plural forms as well, unless the context clearly indicates otherwise. Furthermore, the use of the terms a, an, etc. does not denote a limitation of quantity, but rather denotes the presence of at least one of the referenced items.

The use of the terms “first”, “second”, and the like does not imply any particular order, but they are included to identify individual elements. Moreover, the use of the terms first, second, etc. does not denote any order of importance, but rather the terms first, second, etc. are used to distinguish one element from another. It will be further understood that the terms “comprises” and/or “comprising”, or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Although some features may be described with respect to individual exemplary embodiments, aspects need not be limited thereto such that features from one or more exemplary embodiments may be combinable with other features from one or more exemplary embodiments.

In accordance with certain aspects of an embodiment, and with particular reference to the foregoing Figures, a magnetic resonance guided focused ultrasound frame is provided that avoids one or more disadvantages of the prior art. A frame according to at least certain aspects of the invention is engineered to eliminate the need for invasive fixation methods, such as pinning to the skull, thereby significantly reducing patient discomfort and potential complications.

In an exemplary configuration, frame 100 is particularly configured to rigidly attach to a standard INSIGHTEC MRgFUS system. However, frame 100 is likewise adaptable to other configurations where head immobilization may be required, such as stereotactic radiation therapy or to stop movement of a patient's head during diagnostic MRI scanning.

Frame 100 comprises a unique, hinged configuration, allowing it to transition smoothly between an open state and a closed state (the closed state being shown in the Figures). This configuration facilitates easy placement and removal around the patient's head, enhancing comfort and reducing anxiety associated with the setup process. In accordance with certain aspects of an embodiment, frame 100 comprises a body portion and a hinge mechanism. The body portion of frame 100 comprises front hinged right arm 102, front hinged left arm 104, and rear curved arm 106. These elements provide the body of frame 100 a generally ovoid shape, configured to comfortably encircle the patient's head and neck. This shape is likewise conducive to fitting most human head/neck configurations, providing a secure fit without excessive pressure or punctures with pins. Likewise, the hinge mechanism is formed preferably of a right hinge 110 and a left hinge 112 on opposite sides of the body. Right hinge 110 attaches front hinged right arm 102 to rear curved arm 106, while left hinge 112 attaches front hinged left arm 104 to rear curved arm 106. Each of right hinge 110 and left hinge 112 are located on each side of the body of frame 100, the hinges allowing opening and closing without detaching from the MRI table. This feature may be particularly beneficial for quick adjustments and emergency removals.

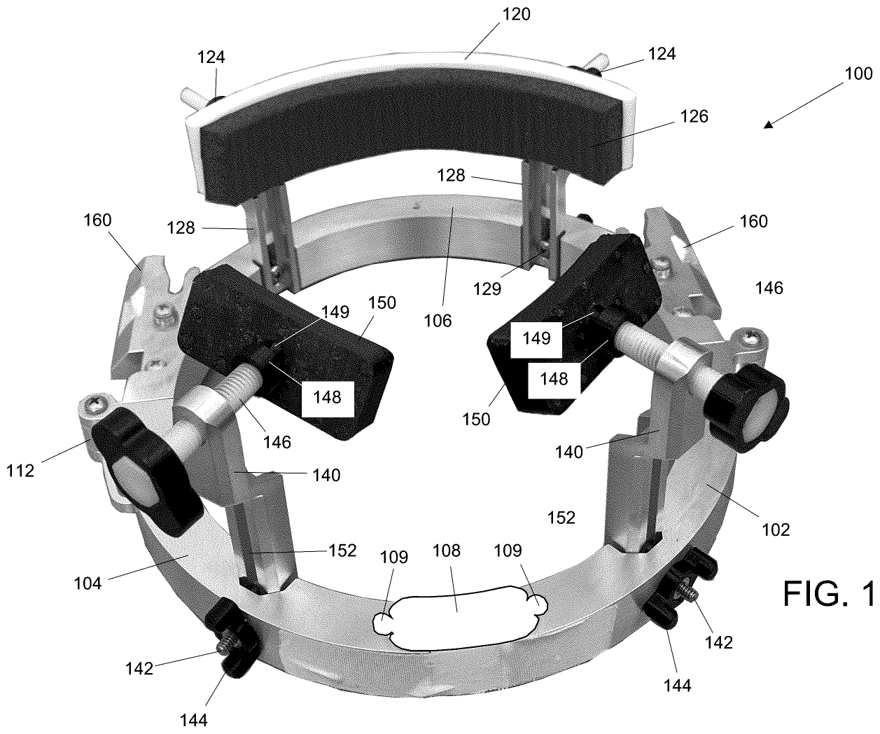

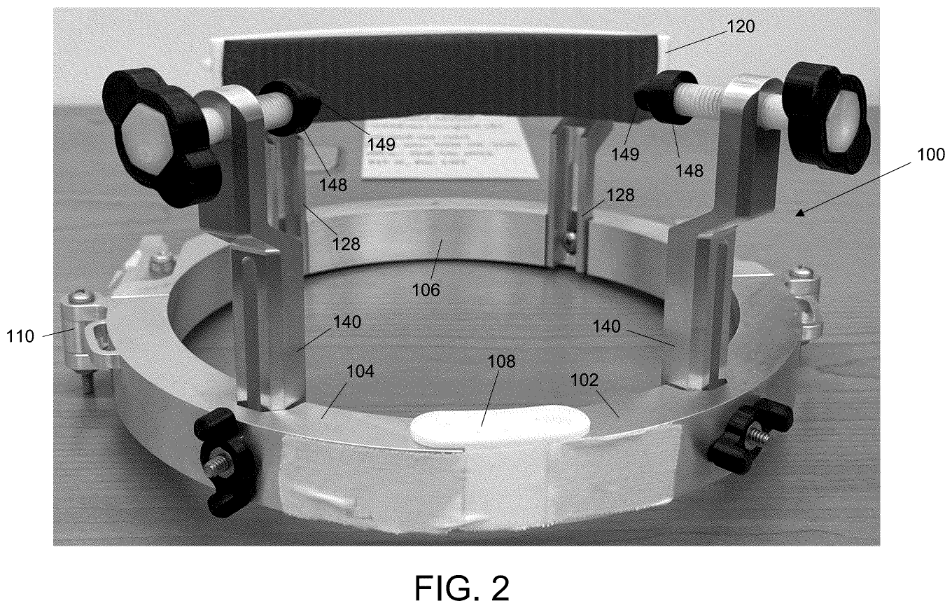

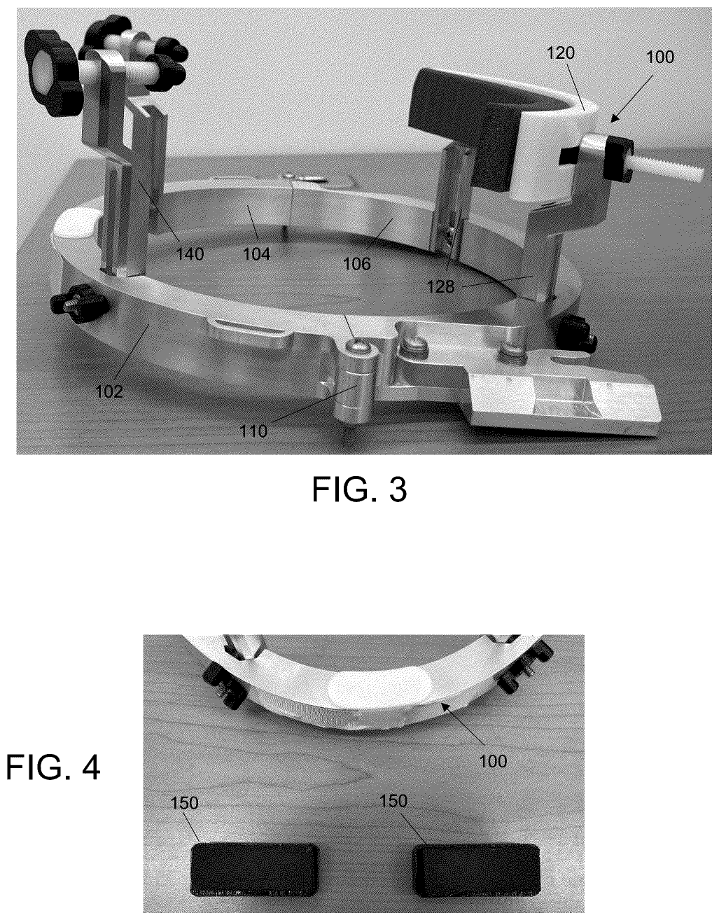

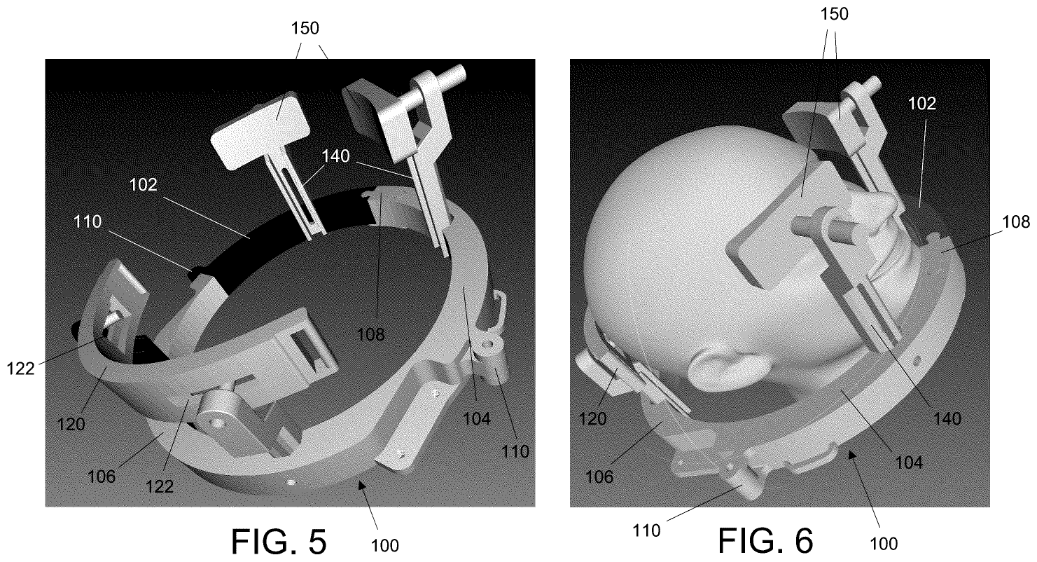

A locking insert 108 is provided that is configured to lock distal ends of each of front hinged right arm 102 and front hinged left arm 104 to one another. As best viewed in FIGS. 1, 5, and 6, locking insert 108 includes interlocking members 109 at its opposing ends, such as a bulb, which are shaped for insertion into similarly shaped openings at the distal ends of front hinged right arm 102 and front hinged left arm 104. When interlocking members 109 are engaged with their mated openings on the body of frame 100, the body of frame 100 is secured in the closed state without the use of traditional screws or pins.

When locked in the closed state, frame 100 may be operated like other existing stereotactic head frames and positioned on the patient while the patient is sitting or standing by, for example, sliding it down from the top of the patient's head. However, in those situations in which it is impractical for the patient to assume a sitting or standing position, the open state may provide significant advantages over standard stereotactic head frames. For example, an open state of frame 100 may be particularly useful in those cases in which the patient cannot actively cooperate in positioning their body, or when the patient is unconscious or critically ill, in cases where the patient is a pediatric patient, and in those cases where the patient feels claustrophobic. Particularly in the case of claustrophobic patients, the open state may reduce the amount of time that the patient must endure the frame while outside of the MRI environment.

Still further advantages may be realized through the use of a hinged frame 100 configured in accordance with aspects of the invention. Specifically, such a hinged frame may ease operation by a single individual with minimal training and may allow for easy termination of procedures and removal of the patient from the MRI environment if necessary. Such situations could be encountered when the patient is claustrophobic, or experiencing nausea or vomiting, experiencing unanticipated adverse events. In these cases, frame 100 may simply be opened from the front, leaving frame 100 attached to the MRI table. The patient's head can move independently from frame 100 for disengagement when necessary. Moreover, the open state of frame 100 may facilitate easy insertion of frame 100 on patients that are hooked to life support equipment and/or intubated and on a ventilator. Such patients may be transferred into the magnet and their head rested on the back rest 120 of frame 100 in the open state. Any tubes or lines associated with the life support or other equipment may the be adjusted appropriately, and frame 100 may then be folded to the closed state around the patient's head and locked in place with locking insert 108.

Arc-shaped back rest 120 provides occipital support in frame 100 and is located at the cranial end of two symmetric, flared hind posts 128. Hind posts 128 are affixed to rear curved arm 106 at their bases, each with a hind post screw 129 and a hind lug nut 130. The patient-facing side of back rest 120 includes a back cushion 126 for patient comfort. A linear back rest articulating window 122 extends along a length of back rest 120 and allows the position of back rest 120 to be adjusted from side to side by loosening back rest clamps 124 on the back side of back rest 120. After adjustment, back rest clamps 124 may be tightened to lock the position of back cushion 126 in place. This configuration may be helpful to increase comfort for the patient during prolonged procedures. Moreover, human head shapes and sizes vary significantly from person to person, and minor side to side adjustments of the position of back rest 120 adds to the optimal head positioning and enhanced comfort distributing pressures more evenly.

Back cushion 126 may comprise a rubber, foam, leather, or similarly configured cushion, providing an extremely comfortable resting surface for the back of the patient's head. A sizable arc of back rest 120 distributes head pressure over a large surface area, preventing the establishment of focal pressure points, discomfort, or injury. Adjusting the position and location of back rest 120 will typically comprise the first step in placement of frame 100 about a patient's head.

Adjustable flared front posts 140 are attached to each of front hinged right arm 102 and front hinged left arm 104 with front post screws 142 and front post lug nuts 144 for supporting the front of the patient's head. Each adjustable flared front post 140 holds a front cushion 150 that contacts the patient's head when frame 100 is placed on the patient's head in the closed position. Front posts 140 are adjustable in height and width, accommodating different head sizes and shapes comfortably while still assuring rigid head fixation. This configuration ensures that frame 100 fits snugly and securely, even without invasive pins, and regardless of the patient's physical characteristics. Front post screws 142 extend through slider openings 152, which allows for sliding movement of the front posts 140 in cranial or caudal directions by simply unscrewing lug nut 144. This significantly ease adjustment for the operator to position front cushions 150 on the patient's forehead (above the level of the patient's eyebrows).

Articulating front post mounts 146 extend through an uppermost portion of each front flared front post 140, and are threaded (along with an internal channel in front posts 140 through which front post mounts 146 pass) to enable threaded adjustment to the radial position of each front post mount 146, and thus of each front cushion 150. Front post mounts 146 thus allow adjustment of frame 100 to meet the skull width of the particular patient. At the end of each articulating front post mount 146, a front post mount cap 148 is provided that is configured to snap into the posterior surface of each front cushion 150. In a particularly preferred configuration, each front post mount cap 148 includes a swivel attachment 149 for engagement with cushion 150, which when snapped into place enables front cushion 150 to be adjusted in angle relative to the flared front posts 140, and thus swivel with respect to the rest of frame 100. This feature in combination with the foregoing mechanisms enabling front post height and width adjustment provides maximum flexibility for the operator to affix frame 100 to the front portion of the patient's head, aligning the front cushions 150 comfortably against the patient's forehead and providing additional stability and comfort.

Front cushions 150 may be formed of foam, rubber, or leather for additional patient satisfaction and comfort. When firmly in contact with the patient's forehead and fixed in place, the patient's head is immobilized and prevented from moving side to side, cranio-caudally, or to rotate. For patients with smaller heads (such as pediatric patients), the height of front posts 140 can be adjusted downward and the articulating post mount 146 moved radially inward, while the reverse may be done for patient's having a larger head diameter, maximizing the usability of frame 100 for patient's of widely varying physiologies.

Frame 100 is constructed from non-magnetic materials to ensure compatibility with MRI environments. For example, frame 100 components (e.g., front hinged right arm 102, front hinged left arm 104, and rear curved arm 106), mounting brackets 160, back rest 120 and back rest posts 128, and front posts 140 are preferably machined from 6061-T6 aluminum, and the back cushion 126, front cushions 150, and locking insert 108 may be preferably 3D printed in Nylon 12, which materials are selected for their non-magnetic properties, light weight, and structural integrity. Such materials reduce the risk of interference with the MRI's magnetic fields and imaging processes. Alternative materials, such as polystyrene, PVC, PU, PTFE, and non-magnetic steel can also be used based on specific requirements such as cost, durability, or additional non-magnetic properties without departing from the spirit and scope of the invention.

Notably, the removeable locking insert 108 locking front hinged right arm 102 and front hinged left arm 104 in the closed orientation in combination with the adjustable back rest 120 and adjustable front cushions 150 (with their associated mounting elements as described above) work cooperatively to ensure that frame 100 provides a stable, secure, and comfortable fit, minimizing any patient head movement during sensitive MR-guided focused ultrasound procedures. The ability to quickly and easily adjust and secure these components not only enhances patient comfort but also improves the efficacy and safety of the medical procedures being performed. Such combination of features provides a robust system for head stabilization, tailored to meet the needs of diverse patient populations and procedural requirements, ensuring both safety and comfort during MR-guided focused ultrasound treatments.

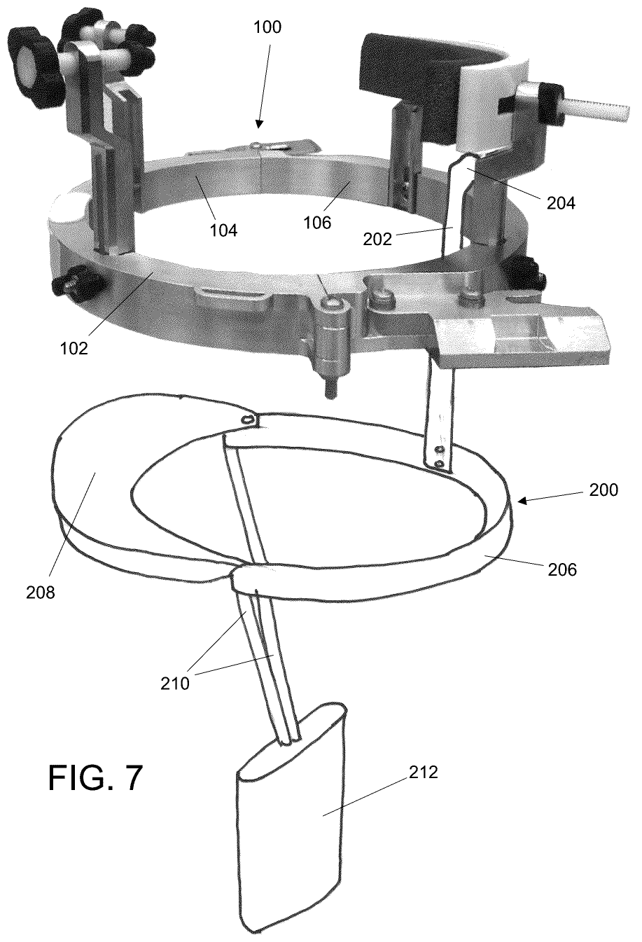

In use, frame 100 may be locked into table mounts for a patient table used during MR-guided focused ultrasound treatments and other operations, such as by way of non-limiting example patient tables used in INGIGHTEC MR-guided focused ultrasound systems. This is achieved through mounting brackets 160 positioned symmetrically on outer opposite sides of rear curved arm 106 of the body of frame 100. Additionally, and with particular reference to FIG. 7, a neck brace assembly 200 may be provided having a posterior neck brace connector 202 that may snap into the posterior aspect of the rear curved arm 106 via a curved hook 204. Neck brace connector 202 preferably consists of a soft plastic back neck collar 206 attached to a flat chin rest 208 configured to hold the patient's chin in place, for example during MR-guided focused ultrasound treatments. Anteriorly and inferiorly are chest piece connectors 210 that articulate with a flat chest piece support 212 configured to be placed on the patient's sternum.

An MR-compatible frame configured in accordance with at least certain aspects of the invention may offer one or more of the following advantages over existing head stabilization devices used in MRI or focused ultrasound procedures that enhance patient comfort, safety, and the overall efficacy of the procedures. Particularly, unlike traditional stereotactic frames that require invasive methods such as pinning directly to the patient's skull, a frame configured in accordance with aspects of the invention may avoid the use of pins to significantly reduce discomfort and potential complications associated with invasive procedures, such as infections and scalp hematoma, thereby enhancing patient safety and comfort. Further, a frame configured in accordance with aspects of the invention includes multiple adjustable components, such as beck rest 120 and front cushions 150, the positions of which can be easily modified to fit various head sizes and shapes. This adjustability ensures a more comfortable and secure fit, which is important for maintaining precise targeting during MR-guided focused ultrasound procedures. The ability to adjust such components quickly and easily also reduces setup time, making the procedure more efficient. Still further, a frame configured in accordance with aspects of the invention may be formed from non-magnetic materials that make it fully compatible with MRI environments. This ensures that the frame does not interfere with the magnetic fields of the MRI, maintaining the quality of imaging and the effectiveness of the focused ultrasound treatment. Still further, a frame configured in accordance with aspects of the invention may include a unique locking mechanism that simplifies the process of securing the frame but likewise enhances patient comfort by eliminating the need for more cumbersome and potentially uncomfortable securing methods, and that provides a quick-release feature that allows for rapid disengagement in emergency situations, further enhancing patient safety. Still further, a frame configured in accordance with aspects of the invention provides a versatile configuration that allows use for a variety of patients regardless of condition (including, for example, those that are unconscious or unable to cooperate in the procedure). Finally, a frame configured in accordance with aspects of the invention may facilitate its use in multiple, repeated usage sessions, which may be necessary in treatments for chronic conditions or in research settings. This can lead to better patient compliance and more consistent treatment outcomes.

In summary, such an MR-compatible frame thus offers significant advancements in patient comfort, adjustability, and MR compatibility over traditional stereotactic frames used in MRgFUS procedures. By addressing the critical limitations of existing devices, a frame configured in accordance with aspects of the invention broadens the applicability of MRgFUS technology, potentially improving treatment outcomes for adverse range of neurological conditions.

### Experimental Testing and Results

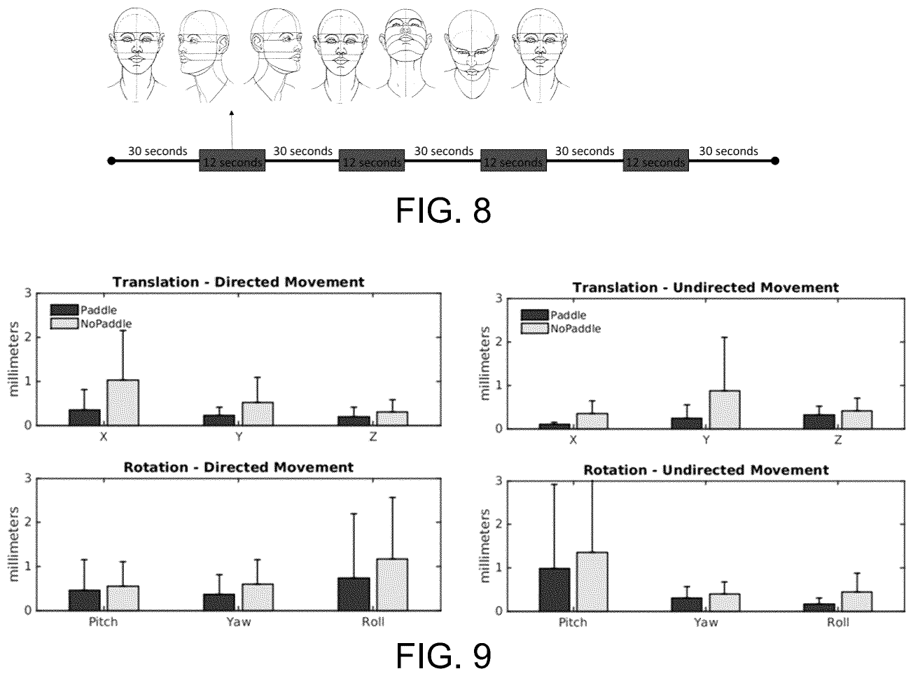

After obtaining an Institutional Review Board approval, a prototype frame configured in accordance with the foregoing description was tested for its safety and efficacy in normal human volunteers. The experimental design and data analysis from six initial subjects is shown in FIG. 8. During periods in which head movement was instructed (intermediate blocks along the timeline), participants recreated the head movements in the order displayed. During the 30 second blocks interleaving movement periods, participants were instructed prior to the scan to remain still.

### Experimental Design:

Participants were asked to complete a simple head movement task to measure the effectiveness of the MRgFUS frame device described above (FIG. 8). During this task, participants were directed to move their head at four different points in the run. Directions to rotate the head were delivered by MRI-compatible headphones and consisted of 6 directions always presented in the following order: 1) rotate head right, 2) rotate head left, 3) return head to neutral position, 4) rotate chin up, 5) rotate chin down, and 6) return head to neutral position. These periods of movement lasted a total of 12 seconds, with each set of directions spaced by 2 seconds.

Interleaved with these movement periods were five, 30 seconds periods where no directions were provided. During this period, participants were instructed to remain still and keep their head fixated as best as possible.

This movement run was completed four times: twice with the head paddles fixed in place and twice with the head paddles not fixed in place (but the frame still in place). Data Processing:

For the entirety of each movement run, head movement in the 6 canonical directions (3 translation, 3 rotation) were computed using SPM. To convert rotation movements (pitch, yaw, roll) from radians to mm, the obtained rotation estimates were projected onto a head of average size (radius=90.71 mm).

The resulting movement matrices were then further divided into periods associated with directed movement and undirected movement. For both the directed and undirected periods, average head movement was calculated across a directed/undirected as the absolute difference between the head position at the start of the period with all other timepoints within the period:

$$Movement = {\sum }_{n = 2}^{N} \frac{\vert {y}_{n} - {y}_{1} \vert}{N}$$

where y is the movement estimate for the n^{th} TR in a directed/undirected movement period and N is the total number of TRs within a period (6 for directed, 15 for undirected).

For each participant, a single set of movement estimates (1 for each direction) for the directed and undirected periods was calculated by averaging the 8 directed and 10 undirected estimates collected for the runs with and without pads.

### Statistical Analysis:

Analysis focused on full datasets collected for 6 participants. The primary aim was to evaluate whether the frame plus pads would significantly reduce movement below 2 mm. This was done using a one sample t-test comparing the observed values against 2. This analysis was completed for both the directed and undirected periods.

For completeness, qualitative data is also provided regarding head movements when the pads were not in place. In general, head movements were always smaller with the pads, but these differences were not significant owing to our relatively small sample size (n=6). The results are graphically depicted in FIG. 9, showing estimates of head movement (mean+1 standard deviation) during the directed (left panel) and undirected movement periods (right panel). Estimates for when the head pads were in place are shown in the left vertical bars of each pair, while estimates obtained without head paddles are shown in the right vertical bars of each pair.

Results for the directed and undirected movement periods are presented as well. For the directed movement periods with the pads in place, movement was significantly lower than 2 mm for X (mean=.34, s.d.=.47, t=−8.65 p<.01), Y (mean=.34, s.d.=.22, t=−18.48 p<.01), Z (mean=.20, s.d.=.22, t=−20.04 p<.01), Pitch (mean=.46, s.d.=70, t=−5.39 p<.01), and Yaw movements (mean=.36, s.d.=.45, t=p=), Z (mean=.20, s.d.=.22, t=−8.73 p<.01). Roll rotations (side-side to rotations) were not found to be significantly less than 2 mm (mean=.73, s.d.=1.46, t=−2.13, p=.08) but were also not found to be significantly greater than 2 mm.

For the undirected movement periods with the pads in place, movement was significantly lower than 2 mm for the X (mean=.10, s.d.=.05, t=−93.08, p<.01), Y (mean=.23, s.d.=.32, t=−13.55, p<.01), Z (mean=.32, s.d.=.20, t=−20.58, p<.01), yaw (mean=.29, s.d.=.27, t=−15.51, p<.01), and roll directions (mean=.16, s.d.=.14, t=−32.19, p<.01). Pitch rotations (nose up and down) were not significantly different from 2 mm (mean=.98, s.d.=1.94, t=−1.29, p=.25) but were also not found to be significantly greater than 2 mm.

It is anticipated that expanding capabilities to perform “pinless” MRgFUS therapies using methods and systems as disclosed herein will have a substantial impact on expanding the use of this technology for cognitive and behavioral neurologic disorders as well as increase the comfort and study compliance in patients with malignant gliomas or neurodegenerative disorders that require multiple repeated BBBD sessions during their treatment cycles. Frames configured in accordance with aspects of the invention may play a role in: (i) addition of new clinical trials and protocols for BBBD (such as AD, ALS, gliomas, drug addiction); (ii) new clinical trials for neuromodulation (potential applications include but are not limited to neuropathic pain, epilepsy, drug addiction, major depression and OCD); and (iii) brain mapping for greater understanding of neuronal circuits in thalamus and basal ganglia

Having now fully set forth the preferred embodiments and certain modifications of the concept underlying the present invention, various other embodiments as well as certain variations and modifications of the embodiments herein shown and described will obviously occur to those skilled in the art upon becoming familiar with said underlying concept. Thus, it should be understood, therefore, that the invention may be practiced otherwise than as specifically set forth herein.

What is claimed is:

1. An MR-compatible frame for use in MR-guided focused ultrasound procedures, comprising:
an ovoid body having a rear arm, a front left arm, and a front right arm;
a first hinge affixing a first end of the front left arm to a first end of the rear arm;
a second hinge affixing a first end of the front right arm to a second end of the rear arm; and
a locking insert configured to simultaneously engage a second end of the front left arm and a second end of the front right arm.

2. The MR-compatible frame of claim 1, wherein the first hinge and the second hinge are configured to transition the frame between an open state and a closed state.

3. The MR-compatible frame of claim 1, wherein the frame is formed from non-magnetic materials.

4. The MR-compatible frame of claim 1, further comprising:
one or more adjustable hind posts; and
a back rest mounted to the one or more adjustable hind posts;
wherein the back rest is adjustable on the hind post from side to side.

5. The MR-compatible frame of claim 4, wherein the one or more adjustable hind posts are adjustable in height from the ovoid body.

6. The MR-compatible frame of claim 4, the back rest having a window extending through the back rest, and wherein a back rest clamp moveably engages the window to enable releasable and selective positioning of the back rest with respect to the one or more adjustable hind posts.

7. The MR-compatible frame of claim 1, further comprising:
a first front post adjustably mounted to the front hinged right arm, and a first front cushion attached to the first front post;
a second front post adjustably mounted to the front hinged left arm and a second front cushion attached to the second front post;
wherein each of the first front post and the second front post is adjustable in height from the ovoid body.

8. The MR-compatible frame of claim 7, wherein each of the first front cushion and the second front cushion is attached to the first front post and the second front post, respectively, via a swivel attachment.

9. The MR-compatible frame of claim 1, wherein opposite ends of the locking insert each define an interlocking member shaped to engage with a mating interlocking receiver on each of the second end of the front left arm and the second end of the front right arm.

10. The MR-compatible frame of claim 1, further comprising a mounting bracket attached to an outer edge of the ovoid body, wherein the mounting bracket is configured for locking engagement with a patient support table.

11. A method for immobilizing a patient's head during an MR-guided focused ultrasound procedure, comprising the steps of:
positioning a patient's head in an MR-compatible frame, wherein the MR-compatible frame comprises:
an ovoid body having a rear arm, a front left arm, and a front right arm;
a first hinge affixing a first end of the front left arm to a first end of the rear arm;
a second hinge affixing a first end of the front right arm to a second end of the rear arm; and
a locking insert configured to simultaneously engage a second end of the front left arm and a second end of the front right arm; and
after positioning the patient's head in the MR-compatible frame, adjusting the frame from an open state to a closed state by closing the front left arm and the front right arm around a front of the patient's head.

12. The method of claim 11, wherein the frame is formed from non-magnetic materials.

13. The method of claim 11, further comprising the step of:
after adjusting the frame from an open state to a closed state, engaging the locking insert with the second end of the front left arm and the second end of the front right arm to lock the frame in the closed state about the patient′ head.

14. The method of claim 11, wherein the frame further comprises a back rest movably mounted to the frame, the method further comprising the step of adjusting a height of the back rest with respect to the ovoid body.

15. The method of claim 11, wherein the frame further comprises a back rest movably mounted to the frame, the method further comprising the step of adjusting a position of the back rest from side to side.

16. The method of claim 11, wherein the frame further comprises a plurality of front cushions movably mounted to the frame, the method further comprising the step of adjusting an angle of a radially interior face of each front cushion with respect to the ovoid body.

17. The method of claim 16, further comprising the step of adjusting a height of each front cushion with respect to the ovoid body.

18. The method of claim 11, wherein the ovoid body further comprises a mounting bracket attached to an outer edge of the ovoid body, the method further comprising the step of temporarily affixing the frame to a patient support table.
